(19) United States
(12) Patent Application Publication
(10) Pub. No.: US 2008/0095828 A1
(43) Pub. Date: Apr. 24, 2008

(54) CLEANING SUBSTRATES WITH COMBINATIONAL ACTIVES

(76) Inventors: Marc Privitera, Walnut Creek, CA (US); David Jackson Lestage, Livermore, CA (US); Gregory Van Buskirk, Danville, CA (US); Robert Iliff, Pleasanton, CA (US); Nikhil Dani, Pleasanton, CA (US); David Strack, Pleasanton, CA (US); William Ouellette, Livermore, CA (US); Jason White, Pleasanton, CA (US); Christina M. Borgese, San Ramon, CA (US)

Correspondence Address: THE CLOROX COMPANY, P.O. BOX 24305, OAKLAND, CA, 94623-1305, US

(21) Appl. No.: 11/550,726

(22) Filed: Oct. 18, 2006

(51) Int. Cl. A61K 9/70

(52) U.S. Cl. 424/443; 510/130

(57) ABSTRACT

Various embodiments of the invention provide a method for treating a surface using a cleaning substrate. The cleaning substrate comprises a nonwoven web. The nonwoven web includes hydrophilic materials and chemical actives. The cleaning substrate provides a controlled release of the chemical actives on the surface to be cleaned. Additionally, the cleaning substrate provides a controlled and enhanced foam delivery to the surface being treated.

### FIELD OF THE INVENTION

The present invention relates generally to cleaning substrates, and more particularly, to cleaning substrates comprising a nonwoven web.

### BACKGROUND OF THE INVENTION

Cleaning substrates comprising a nonwoven web have been used to treat various types of products and/or surfaces. The cleaning substrates comprise nonwoven webs impregnated with absorbent materials. Nonwoven materials are commonly used in numerous in cleaning devices and health care products such as surgical drapes, gowns and bandages, infant, child and adult personal care absorbent products such as diapers, swimwear, incontinence garments and pads, sanitary napkins, sanitizers, floor cleaners, wipes, and so forth.

Nonwoven materials are frequently used in personal care products, health care products and cleaning devices because they may be highly absorbent, relatively inexpensive and disposable. For these reasons, many of the nonwoven products that are impregnated with absorbent materials are used to remove unwanted fluids or soils from a surface and to dispose of the used nonwoven absorbent material. Suitable examples of disposable absorbent nonwoven products include paper towels, facial wipes, diapers, etc. In these common absorbent materials, any added absorbent particles or fibers are generally used to increase the absorbent capacity of the nonwoven material.

### DESCRIPTION OF RELATED ART

U.S. Patent Application Number 2005/0165376A1, entitled ‘Designing Dry and Porous Absorbent Composites Containing Super-Absorbent Polymers’, by Buchholz et al. describes super-absorbent polymer composites and a method for designing such composites. The method involves intermixing permeable substruction meshwork strands and super-absorbent polymer particles. This produces a meshwork, which absorbs a predefined mass of liquid to reach a specified dryness quality, and porosity.

U.S. Patent Application Number US2005/031852, entitled ‘Absorbent Article Comprising Coated Water-Swellable Material’, by Schmidt et al. describes an absorbent structure containing water-swellable materials. The absorbent structure comprises a nonwoven web. The water-swellable materials are hydrogel-forming polymers having a coating, which prevents the polymers from rupturing in the presence of water or saline water.

WO2002091976, entitled ‘A Process for Making a Fibrous Web Containing a Functional Material’, assigned to the Procter and Gamble Company describes a process of forming a fibrous web. The fibrous web includes fibers and a functional material, such as an odor control material, and/or an antimicrobial material. The fibers are bonded together by a binder, and the binder immobilizes the functional material. Further, the patent publication describes disposable absorbent articles comprising the odor control and/or antimicrobial material.

U.S. Pat. No. 5,483,949A, titled ‘Exothermic Compositions and Container for Heating Food’ by Dean describes exothermic compositions for warming a sealed food container. The exothermic compositions have a long shelf life and generate heat on contact with water. Further, the patent publication describes an exothermic reaction between an acid, added to exothermic composition, and lime for generating heat and leaving only a neutral residue.

The above-mentioned patent publications relate to nonwoven web and their methods of preparation. Further, the above-mentioned patent publications are more specifically focused on the containment of fluids and active chemicals. However, they do not describe the controlled release of active chemicals to a target site. Further, they also do not describe attenuating the heat generated in an exothermic reaction between the active chemicals and a solvent, which may be required for better cleaning, or any other heat enhanced functionality.

There exists a need for a cleaning composition comprising nonwoven web, which can provide time-based release of active chemicals to a target site. Also, there is a need for a nonwoven web that can release active chemicals at a relatively stable concentration profile relative to the target. Further, there is a need for attenuating the heat generated as a result of the reaction between the active chemicals and the solvent.

### SUMMARY OF THE INVENTION

Embodiments of the invention provide a cleaning substrate and a method for treating a surface using a cleaning substrate. The cleaning substrate of the present invention comprises a nonwoven web with one or more layers of nonwoven material that may be in the form of roll stock or randomly intermingled roll stock. The roll stock contains an active chemical or a mixture of one or more active chemicals. Examples of active chemicals include antimicrobials, disinfectants, heat-generating chemicals, sanitizers, etc. Additionally, the nonwoven web may include one or more hydrophilic materials. The incorporation of hydrophilic materials such as sodium polyacrylate, superabsorbing polymer particles like clay, starch, etc. in the nonwoven web. The active chemicals and hydrophilic materials may be incorporated into the nonwoven web by a wide variety of methods.

An embodiment of the invention provides a method for treating a surface using a cleaning substrate, in accordance with an embodiment of the invention. The method involves forming the cleaning substrate. The cleaning substrate comprises a nonwoven web. Hydrophilic materials and chemical actives are incorporated into the nonwoven web. The method further involves exposing the surface of the cleaning substrate to a solvent; allowing the cleaning substrate to absorb the solvent; and creating a spatial interaction between the hydrophilic materials and the chemical actives. The spatial interaction is created such that the hydrophilic materials compete with the chemical actives for the solvent, which provides a controlled release of the chemical actives to the surface being cleaned.

Another aspect of an embodiment of the invention, provides a cleaning substrate for controlled delivery of chemical actives to a surface. The cleaning substrate comprises a nonwoven web, which includes hydrophilic materials and chemical actives. The spatial interaction between the hydrophilic materials and the chemical actives allows the chemical actives to have a gradual release profile. A gradual release profile means that the chemical actives are released from the cleaning substrate of the present invention are released in a slower, steadier manner than they would be from a cleaning substrate without a hydrophilic materials or where there is no spatial interaction between the hydrophilic materials and the chemical actives.

Yet another aspect of an embodiment of the invention, provides a cleaning substrate for controlled delivery of chemical actives to a surface. The cleaning substrate comprises a nonwoven web, which includes hydrophilic materials and chemical actives. The spatial interaction between the hydrophilic materials and the chemical actives enables the substrate to provide a controlled and long-lasting foam delivery to the surface being treated.

A method for treating a surface using a cleaning substrate in accordance with the embodiments of the invention enables a controlled release of chemical actives to the target site. In accordance with an embodiment of the invention, the nonwoven web has time-based holding and releasing properties. These enhance the efficacy of the chemical actives by manipulating the contact time for spatial interaction between the chemical actives and the hydrophilic materials. Hydrophilic materials, including but not limited to, superabsorbent polymers, absorbent clays, and starch, absorb water and make the water available to the chemical actives at a and relatively steady rate which enables the chemicals to be released from the nonwoven in a controlled manner. Further, this allows enhanced efficacy at lower concentration of the chemical actives to be presented to the target because the majority of the chemical actives are not immediately released during the initial wetting of the nonwoven web. The invention also enables the chemical actives to be released at a relatively stable concentration profile relative to the target.

In accordance with various embodiments of the invention, the nonwoven web retains sufficient structural integrity for mechanical cleaning or scrubbing of a surface. Additionally, the nonwoven web may be designed to provide low linting, dust and residue levels. Hydrophilic materials like superabsorbent polymers provide a mechanism for holding moisture, which may later be made available to other functional chemical actives. The invention also enables the formation of different laminate structures by varying the amount of the chemical actives and the length of their functional activity at the point of use.

In one embodiment, the incorporation of the hydrophilic materials into the nonwoven web may enable the attenuation of the exothermic reaction between the chemical actives and the solvent. Heat attenuation may be controlled by manipulating the particle size of the chemical actives and the hydrophilic materials. Even though the heat generation is attenuated, it is still sufficient to provide warmth to the consumer's skin on using the cleaning substrate or the heat to the surface being cleaned to aid in the cleaning process.

The cleaning substrate, in accordance with the various embodiments of the invention, can be used to clean a wide variety of surfaces. The chemical actives incorporated in the nonwoven web, include but are not limited to, antimicrobials, disinfectants, heat generating chemicals, sanitizers, surfactants, detergents, and naturally derived antimicrobial substances such as creosote bush, rosemary oil, pine oil, natural essences, and other suitable cleaning and sanitizing agents.

The cleaning substrate may be used for the removal of films from the surfaces of a wide variety of items, including but not limited to, refrigerators, televisions, palm computers, laptops, and computer monitors. The cleaning substrate can also be used to clean hard surfaces, including but not limited to, bathroom surfaces (e.g. floor, tub, shower, mirror, glass, toilet seats, etc.); kitchen surfaces (e.g. counter tops, stoves, ovens etc.), and furniture surfaces (e.g. tables, chairs, sofas, etc.). The cleaning substrate may also be useful in the cleaning of hardwood floors, tiles, ceramic, stone, marble, windows, window ledges, tools, automobiles, medical and/or dental equipment, etc.

In accordance with an embodiment of the invention, the cleaning substrate may also be used in a variety of places that require regularly cleaning and disinfecting, including but not limited to, retail facilities, educational facilities, industrial and manufacturing facilities, office premises, hotels, restaurants, theaters, and other places of entertainment and health care. In accordance with another embodiment of the invention, the cleaning substrate may be used to form a functional protective layer on surfaces, including but not limited to, litter boxes, animal cages, puppy pads, counter tops, pantry shelves, refrigerator shelves, bathroom surfaces and other suitable surfaces.

Other aspects and advantages of the present invention will become apparent to one ordinarily skilled in the art from the following descriptions, in conjunction with the accompanying drawings provided herein.

### BRIEF DESCRIPTION OF THE DRAWINGS

FIG. 1 is a flowchart illustrating a method for treating a surface using a cleaning substrate, in accordance with an embodiment of the invention;

FIG. 2 shows multiple configurations of chemical actives and hydrophilic materials in parallel planes, in accordance with various embodiments of the invention;

FIG. 3 is a graph illustrating Magnesium Sulfate and Sodium Bentonite attenuation curves, in accordance with an embodiment of the invention; and

FIG. 4 is a graph illustrating heat release of different ratios of chemical actives with superabsorbing polymers (SAP), in accordance with an embodiment of the invention.

### DETAILED DESCRIPTION OF THE INVENTION

The use of the terms “a,” “an,” “the,” and similar articles in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise indicated.

Various embodiments of the invention describe a method for treating a surface using a cleaning substrate or article. The cleaning substrate comprises of a nonwoven web, chemical actives incorporated into the nonwoven web, and hydrophilic materials incorporated into the nonwoven web.

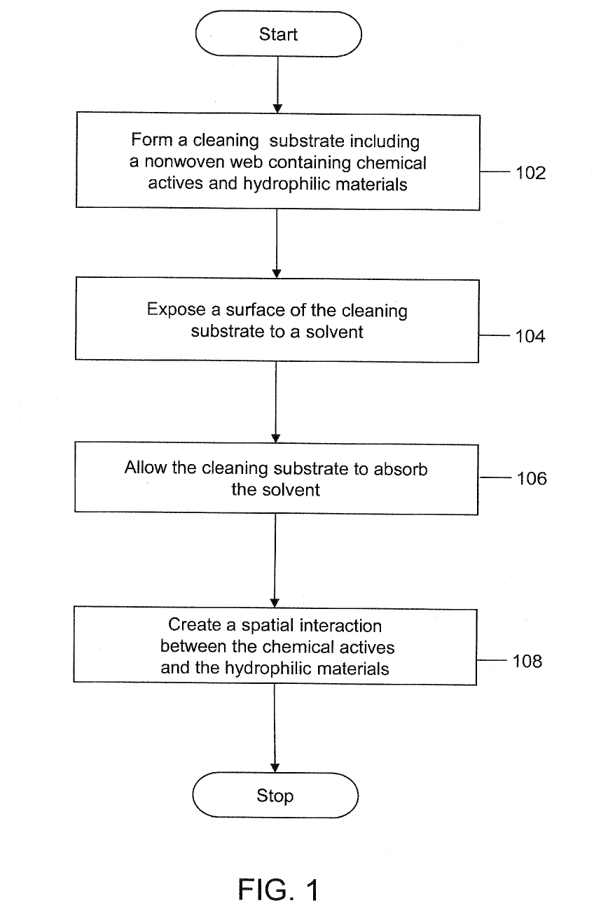

FIG. 1 is a flowchart illustrating a method for treating a surface using a cleaning substrate, in accordance with an embodiment of the invention. At step 102, the cleaning substrate is formed. The cleaning substrate is composed of a nonwoven web. The hydrophilic materials and the chemical actives are incorporated into the nonwoven web.

In accordance with an embodiment of the invention, the nonwoven web may include nonwoven fibrous sheet materials. In accordance with another embodiment of the invention, the nonwoven web includes, but is not limited to, meltblown, coform, air-laid, spun-bound, wet-laid, bonded-carded web materials, and/or hydro entangled, and spun-laced materials.

In accordance with various embodiments of the invention, the hydrophilic materials include, but are not limited to, superabsorbing polymers, silica, collagen, pectin, gelatin, starches, guar gum, gum arabic, locust bean gum, gum karaya, alginic acid, and sodium and calcium salts, and combinations thereof. The superabsorbing polymers (SAP) can be in the form of particles (e.g., granules, flakes, inter-particle aggregates, interparticle crosslinked aggregates, and the like). The SAP can also be in the form of fibers, sheets, films, foams, laminates, and the like. The hydrophilic materials may be water-swellable gelling polymers. Examples of the water-swellable gelling polymers, include but are not limited to, polysaccharides such as carboxymethyl starch, carboxymethyl cellulose, and hydroxypropyl cellulose; nonionic types such as polyvinyl alcohol, and polyvinyl ethers; cationic types such as polyvinyl pyridine, polyvinyl morpholine, N,-dimethylaminoethyl, N,-diethylaminopropyl acrylates, methacrylates, and the respective quaternary salts thereof.

In accordance with one embodiment of the invention, the hydrophilic material may be a synthetic substance. Exemplary synthetic substances include, but are not limited to, sodium carboxymethyl-cellulose, polyvinyl alcohol, polyvinyl pyrollidone, polyethylene glycols, crosslinked dextran, starch acrylonitrile graft copolymer, starch sodium polyacrylate, gluten, polymer of methyl vinyl ether and maleic acid and derivatives, polyvinyl pyrrolidone, polyethylene glycols, polypropylene glycols, metals and ammonium salts of polyacrylic acid, or copolymers thereof, or combinations thereof.

The chemical actives incorporated into the cleaning substrate, inlcude but are not limited to, antimicrobials, disinfectants, heat generating chemicals, sanitizers, surfactants and/or detergents. Exemplary chemical actives include, but are not limited to, sodium bicarbonate, citric acid, carbon, sodium hypochlorite, quaternary ammonium salts, chitosan, and any hypocholorus producing agents. The chemical active may be a naturally derived antimicrobial substance like creosote bush, rosemary, pine oil, any other essential oil or suitable plant extracts. The chemical actives may include biguanide compounds such as polyhexamethylene biguanide hydrochloride, p-chlorophenyl biguanide, 4-chlorobenzhydryl biguanide, chlorhexidine and its salts. The chemical actives may also include alcohols, peroxides, boric acid and borates, chlorinated hydrocarbons, organometallics, halogen-releasing compounds, mercury compounds, metallic salts, organic sulfur compounds, iodine compounds, silver nitrate, quaternary phosphate compounds, and phenolics.

At step 104, the surface of the cleaning substrate is exposed to a solvent. In accordance with one embodiment of the invention, the solvent is water. Water can include tap water, filtered water, bottled water, spring water, distilled water, deionized water, industrial soft water, any other suitable sources of water. The de-ionized water and/or the industrial soft water can reduce the amount of residue formation and also reduce the amount of undesirable metal ions.

After exposing the cleaning substrate to the solvent, at step 106, the cleaning substrate absorbs the solvent. The hydrophilic materials incorporated in the nonwoven web are used to absorb and contain the solvent. This allows controlled dissolution and/or controlled reaction of the chemical actives with the solvent in the nonwoven web. A combination point may be in the process step that allows the final nonwoven web structure to be formed, it may be prior to use or it could include the actual point of use so that the functional efficacy is at its peak during the desired usage of the cleaning substrate.

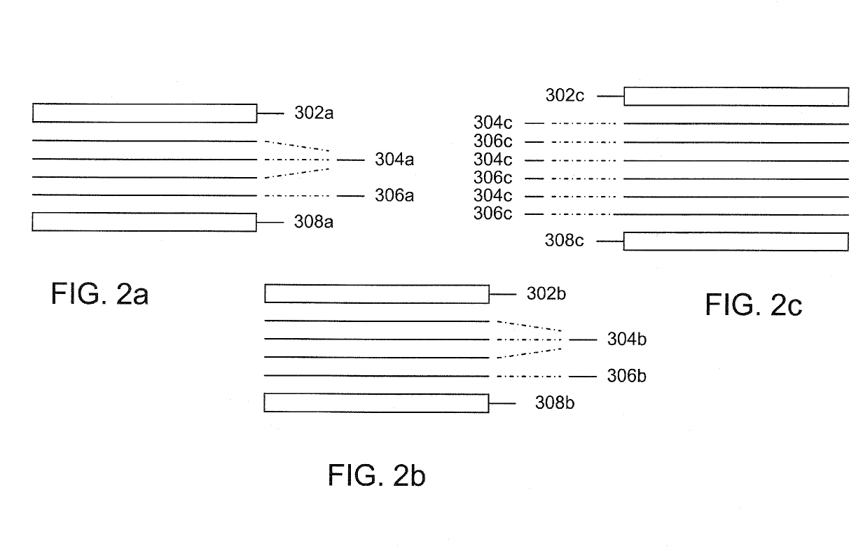

At step 108, a spatial interaction between the chemical actives and the hydrophilic materials is created. The creation of the spatial interaction between the chemical actives and the hydrophilic materials can occur in a variety of ways. In one embodiment of the invention, the chemical actives and the hydrophilic particles can be held in parallel layers to create sufficient proximity for spatial interaction. This is further illustrated in conjunction with FIG. 2. For example, the nonwoven web comprising two or more layers may include the chemical actives in one layer and the hydrophilic materials in an adjacent layer. In one aspect of the embodiment of the invention, the cleaning substrate includes an abrasive layer. In another aspect of the embodiment of the invention, the cleaning substrate includes a hi-loft layer.

In accordance with another embodiment of the invention the chemical actives and the hydrophilic particles can be held in a random blended proximity to create a spatial interaction which means having the hydrophilic particles in sufficient proximity so that compete with the chemical actives when they are exposed to a solvent. The chemical actives incorporated into the nonwoven web, require the spatial interactionbetween the hydrophilic particles and chemical actives so that the release rate of the actives may be controlled.

The chemical actives may serve a variety of purposes, for example, when a surface soil is being treated, the surface soil is exposed to a concentration of chemical active that may dissolve, loosen and/or physically abrade the soil thereby helping its release from the surface to which it connects. The chemical actives can also be used for disinfecting a surface from microorganisms. This requires the surface to be exposed to a certain concentration level of the chemical actives, which is easier to accomplish with a cleaning substrate that controls the release of active chemicals. The disinfecting mechanism depends on a contact time between the chemical actives and the targeted microorganisms. The controlled release of actives also enables the cleaning substrate be an effective disinfecting cleaning tool for longer periods of time because of the controlled release of active chemicals.

In accordance with an embodiment of the invention, the spatial interaction of the chemical actives and the hydrophilic particles allows the chemical actives to have a gradual release profile. A gradual release profile or controlled release means that there is a substantially slow and steady release of actives over time. Generally, when the solvent contacts a standard cleaning substrate with actives, a spike in the concentration is observed at the targeted area, and then the remaining chemical active is diluted and moved from the target due to salvation and mechanical surface fluid dynamics of the target. However, when the chemical actives are incorporated along with the hydrophilic materials in the nonwoven web, the solvent is taken up and held so that the chemical actives can be released at a substantially stable concentration profile, relative to the target.

This gradual release profile provides an advantage over a standard cleaning substrate impregnated with actives because many of the actives are released at the initial exposure to the solvent which creates an excess of actives at the beginning of use and a sharp decrease in actives over time. Since many of the actives are released at the beginning of use for a standard cleaning substrate there are fewer actives available to be released later in use which results less cycles where the cleaning substrate can effectively release actives for cleaning surfaces.

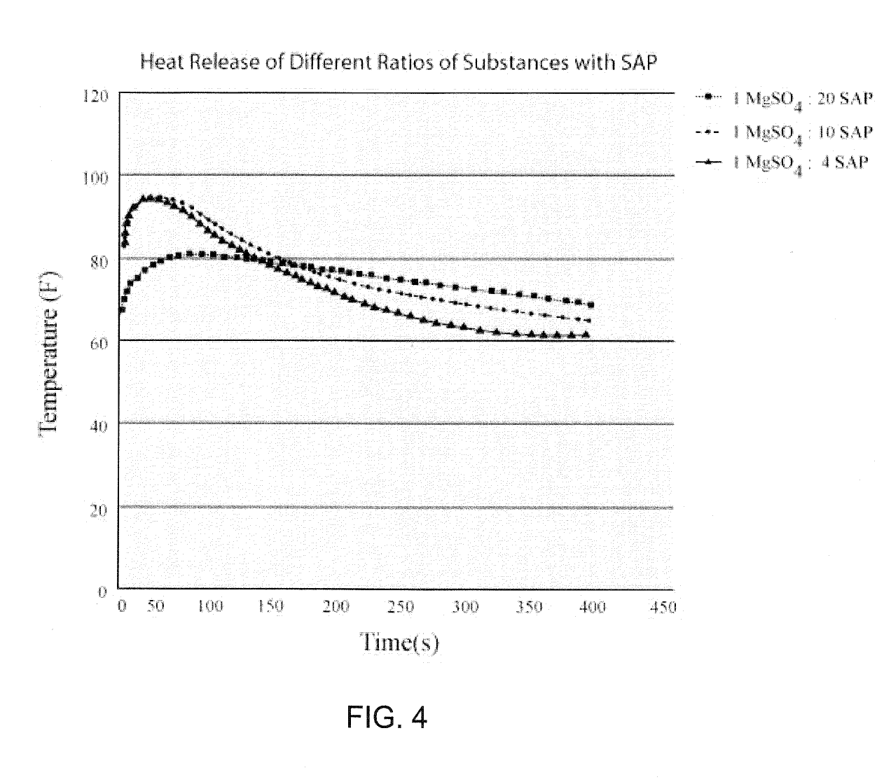

In accordance with another embodiment of the invention, the reaction between the chemical actives and the solvent generates heat that can be controlled over a period of time. The incorporation of the hydrophilic materials in the nonwoven web leads to exothermic attenuation as the hydrophilic materials compete with the actives for solvent. The nonwoven web solid particles blended with solid particles in a random walk type low shear solid blending system, which is not enough to degrade the particles. Further, the particle size of one or more chemical actives and hydrophilic materials may be varied to refine exothermic attenuation. The exothermic attenuation characteristics may vary depending on the ratio of the chemical actives to the hydrophilic materials. This is described in detail in conjunction with FIG. 4.

FIG. 2 illustrates multiple configurations of chemical actives and hydrophilic materials, in accordance with various embodiments of the invention.

As illustrated in FIG. 2a, the nonwoven web may have a six-layer configuration. In FIG. 2a, layer 302a is a hi-loft layer. Layers 304a are three layers containing alkylpolyglucoside (APG). Layer 306a contains superabsorbent polymer (SAP). Layer 308a is a blue scruby layer. In various embodiments of the invention, any layer of the nonwoven web or combinations of the layers may be colored in one or more colors or comprise an abrasive material.

As illustrated in FIG. 2b, the nonwoven web has a six-layer configuration. Layer 302b is a hi-loft layer. Layers 304b are three layers of the APG. Layer 306b has a combination of SAP and sodium bicarbonate. Layer 308b is a blue scrub layer. The bicarbonate reacts with the water present in the nonwoven web, thus creating carbon dioxide bubbles, which increase the appearance of foam.

As illustrated in FIG. 2c, the nonwoven web has an eight-layer configuration, in which the SAP and the APG layers are arranged in an alternate pattern. Layer 302c is a hi-loft layer. Layers 304c are three layers of the APG. Layers 306c are three layers of SAP. Layer 308c is a blue scrub layer.

The gradual release profile of the chemical actives was tested using a foam mileage test as described below.

### Foam Mileage Test:

Equipment used:
Gardner Abrasion Tester
Plexiglass template with 6″×6″ cut out
Minimum of two 6″×6″ glass tiles
Syringe capable of holding at least 40 cc of water
A square or paper towels to remove excess foam from under Plexiglass template
Assumptions:
1 Rep=back and forth on the Gardner Abrasion Tester
1 Cycle=50 Reps
1 Cycle=16.86 square feet
Area cover=square feet of nonwoven web and glass covered/cycle
Nonwoven web gets wet at the beginning of each cycle
Soil load=1-6″×6″ MUD soap scum bathroom soil/cycle
Light soil coverage
This test can be done with or without soil
Average shower/tub=109 square feet

### Method:

The test procedure included placing a Plexiglas template with two 6″×6″ glass tiles stacked on top of each other. The Gardner Abrasion Tester counter was set to 50 reps. The cover of the counter was closed and the reset button was pressed. The scrubby side was placed down on a glass tile and the syringe was filled with 34 cc for initial wetting. It was dosed on evenly over the nonwoven web to ensure the wetting of the edges. The amount of foam coverage was evaluated visually so that it encompassed the glass tile and was done at the end of 50 reps. The nonwoven web was removed when the Gardner stopped and the hi-loft was placed side down. The water and foam were removed from the Gardner surface. The template and the glass tile were removed of all the surfactant. The two steps of placing and setting of the Gardner count were repeated. During the second and consecutive cycles the amount of blue lotion remaining on the scrubby side was noted. This indicates the presence of the lotion in the absorbent layer. The evaluation was done till the foam failed to appear. The foam failure cycle is not used for determining the square feet covered by the nonwoven web. The calculations are made as follows:

No. of cycles×Sq ft. Example 10 cycles×16.86 square feet=168.6 square feet covered.

168.6 square feet/109 square feet=No. of shower tubs cleaned.

The following non-limiting examples are provided to further illustrate different configurations of the nonwoven web:
Example 1: Hi-loft-APG-SCRUB (blue)
Example 2: Hi-loft-APG-APG-SCRUB (blue)
Example 3: Hi-loft-APG (3)-SCRUB (blue)
Example 4: Hi-loft-APG (4)-SCRUB (blue)
Example 5: Hi-loft-APG (3)-SAP-SCRUB (blue)
Example 6: Hi-loft-(APG-SAP) X3-SCRUB (blue)
Example 7: Hi-loft-APG (3)-SAP/Bicarb-SCRUB (blue)
Example 8: Hi-loft-(APG-SAP/Bicarb) X3-SCRUB (blue)

The results of the foam mileage test for Example 1, Example 6 and Example 8 are set forth in Table 1, Table 2 and Table 3, respectively.

TABLE 1
Example 1: Hi-loft - APG - SCRUB (blue)

| | Hi-loft | | SCRUB | Total |
|---|---|---|---|---|
| Sample | (g) | APG (g) | (blue) g | w/g |
| A | 2.32 | 1.24 | 0.70 | 6.26 |
| B | 1.94 | 1.19 | 1.36 | 6.74 |
| C | 2.34 | 1.28 | 1.38 | 7.00 |
| D | 2.31 | 1.18 | 0.69 | 6.10 |
| E | 2.23 | 1.20 | 0.61 | 6.27 |
| F | 1.91 | 1.17 | 0.66 | 5.95 |
| G | 2.15 | 1.21 | 0.69 | 5.72 |

TABLE 2
Example 6: Hi-loft − (APG − SAP) × 3 − SCRUB (blue)

| | Hi-loft | Total APG | | Total SCRUB | |
|---|---|---|---|---|---|
| Sample | (g) | (g) | Total SAP | (blue) g | Total w/g |
| A | 2.00 | 3.22 | 4.77 | 0.71 | 14.02 |
| B | 2.03 | 3.12 | 4.76 | 0.71 | 13.93 |
| C | 1.90 | 3.52 | 4.85 | 0.73 | 11.74 |
| D | 1.63 | 3.62 | 4.57 | 0.74 | 13.36 |
| E | 2.18 | 3.44 | 4.57 | 1.43 | 13.48 |
| F | 1.68 | 3.18 | 4.50 | 1.41 | 14.07 |
| G | 1.71 | 3.28 | 4.48 | 0.71 | 13.24 |

TABLE 3
Example 8: Hi-loft − (APG − SAP/Bicarb) × 3 − SCRUB (blue)

| | Hi-loft | Total APG | Total SAP | Total SCRUB | Total |
|---|---|---|---|---|---|
| Sample | (g) | (g) | Bicarb | (blue) g | w/g |
| A | 1.93 | 3.72 | 3.75 | 0.72 | 13.07 |
| B | 1.80 | 3.63 | 3.29 | 0.72 | 12.55 |
| C | 1.95 | 3.47 | 3.13 | 0.71 | 12.00 |
| D | 1.68 | 3.54 | 3.44 | 0.71 | 13.11 |
| E | 1.96 | 3.24 | 3.38 | 0.70 | 12.29 |
| F | 2.31 | 3.04 | 3.15 | 0.71 | 10.73 |
| G | 2.10 | 3.52 | 3.56 | 0.71 | 12.67 |

In the above-mentioned examples the scrubby layer is blue in color. However, the invention should not be construed to be limited to only the blue color of the scrub layer. It will apparent to a person skilled in the art that the scrub layer can be of any color.

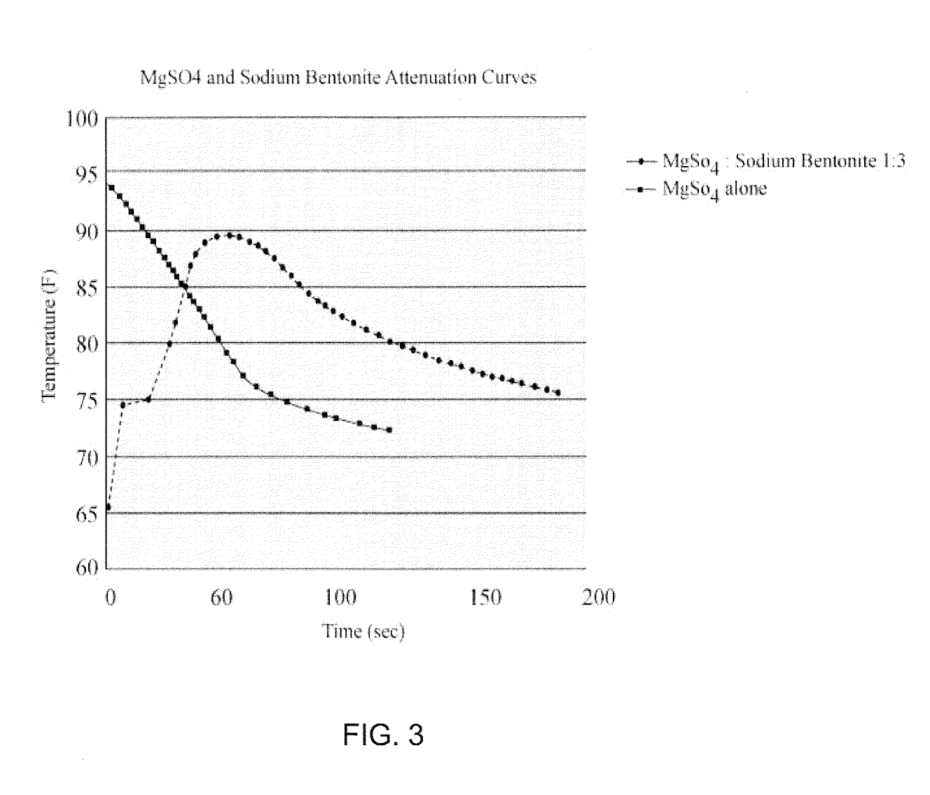

It was observed that Example 6 produced the best results with the foam delivery cycle lasting up to 34 cycles. The foam mileage test for Examples 3 and 7 resulted in foam delivery cycle lasting up to 6 cycles and 18 cycles, respectively. In accordance with various embodiments of the invention, there is a controlled long lasting foam delivery to the surface being treated. Foam appearance is an indication of the release rate of the chemical actives. In accordance with various embodiments of invention, the chemical actives have a gradual release profile, which allows the nonwoven web to release foam during a greater number of cleaning cycles. Thus the graphical representations in FIG. 3 and FIG. 4 show, the release profile of the chemical actives when incorporated along with the hydrophilic materials in the nonwoven web is slowly sloping for longer time as compared to large spike and rapid decline observed in the release profile of the chemical actives without hydrophilic materials in the nonwoven web. Additionally, the chemical actives have a controlled release at a more stable concentration profile relative to the target.

In accordance with various embodiments of the invention, the hydrophilic materials and the chemical actives are bonded by methods including, but not limited to, Thermal Bonding, Through-Air-Bonding (TAB), Needling, Chemical Bonding, Point Bonding, Ultrasonic Bonding and combinations thereof. Point bonding is the main method of binding in the case of disposables like diapers, sanitary products and medical products. The method generally involves heating for a few milliseconds. Bonded areas are completely compressed and dense, and un-bonded areas are open, breathable and porous. The products obtained can be of a very wide parameters ranging from thin, inelastic, strong, stiff, bulky, weak, flexible and extensible. Ultrasonic Bonding generally provides a high amount of softness and breathability to the nonwoven web. The hi-loft layers can also be bonded by Through-Air-Bonding. Through-Air-Bonding involves the application of hot air to the surface of the nonwoven web and results in the formation of products, which are soft, breathable, extensible and absorbent. In a preferred embodiment, during the foam mileage test, different layers of the nonwoven web are bonded with ultrasonic bonding.

Table 4 provides details of different compositions of the nonwoven web.

TABLE 4

| | | | | Thickness | |
|---|---|---|---|---|---|
| | | | Basis | (Thwing | Absorption |
| Layer | Fiber | Manufacturing Process | Weight | Albert) | Capacity |
| Scrubby | Polypropylene | Carded Fibers with a 10 gsm | 100 gsm | 2.08 mm | 680% |
| Layer | 3.12 denier | Spunbond | (Includes | | |
| | | reinforcement | 10 gsm | | |
| | | | Spunbond) | | |
| Absorbent | Polypropylene | 6 Layers Ultrasonically | 320 gsm | 2.32 mm | 360% |
| Layer | | Bonded Together, Will be 4 | | | |
| | | Layers | | | |
| | | 60 gsm Carded PP Thermal | | | |
| | | Bond, Total of 200 gsm | | | |
| | | Spunbond PP, and a 60 gsm | | | |
| | | Carded PP Thermal Bond | | | |
| Hi-Loft | 3.6 denier | Carded Bicomponent with | 138 gsm | 6.1 mm | 780% |
| Layer- times | Bicomponent | Through Air Bonding | | | |
| two | (PE/PET) | | | | |

FIG. 3 is a graph illustrating MgSO₄ and sodium bentonite attenuation curves, in accordance with an embodiment of the invention. The reaction of the chemical actives with the solvent is an exothermic reaction. The addition of the hydrophilic materials to the chemical active in the nonwoven web causes the chemical active (i.e., heat generating particle) to compete for the solvent (i.e., water) with the capillary action of the hydrophilic material (i.e., the water absorbing material), which results in exothermic attenuation. Thus, a rapid temperature spike and then a sharp decline in the temperature is observed when the chemical active is present alone in the nonwoven web, whereas a initial increase in temperature and then a gradual decline in the temperature is observed when the hydrophilic material is added to the chemical active. As shown in FIG. 3, there is a sharp decline in the temperature when the chemical active MgSO4 is present alone in the nonwoven web, whereas there is a gradual decline in the temperature when the hydrophilic material Sodium Bentonite is added to MgSO4.

FIG. 4 is a graph illustrating heat release of different ratios of chemical actives with superabsorbing polymers (SAP), in accordance with an embodiment of the invention. As shown in FIG. 4, there is a gradual decline in the temperature of the reaction between the chemical active and the solvent, when the ratio of the SAP to the chemical active in the nonwoven web increases. This is because more the amount of the SAP incorporated along with the chemical active in the nonwoven web, less is the availability of the solvent available to the chemical active. Hence, there is heat attenuation and a gradual decline in the temperature is observed. The heat attenuation is dependent upon the ratio between the chemical active and the SAP. FIG. 4 shows a quick rise in the temperature followed by a sharp decline in the temperature when MgSO4 and SAP are present in the ratio of 1:4. When MgSO4 and SAP are present in the ratio of 1:10 the heat rise is not very quick and decline in the temperature is less (as compared to when the MgSO4 and SAP are present in the ratio of 1:4). When the ratio of MgSO4 and SAP is 1:20 there is a gradual rise in the temperature followed by a gradual decline in the temperature.

In one embodiment of the invention,the cleaning substrate is used for treating the surfaces of refrigerators, microwaves, counter tops, toilet seats, laminate floors, hardwood floors, tiles, and ceramics etc.

Another embodiment of the invention provides a cleaning substrate, which can be used as a floor cleaner.

Still another embodiment of the invention provides a cleaning substrate that can be used in superabsorbent towels and dispenser rolls.

Yet another embodiment of the invention provides a cleaning substrate that can be used to form a functional and protective layer in litter boxes, animal cages and animal pee pads.

Yet another embodiment of the invention provides a cleaning substrate that can be used in personal care or medical applications to clean and/or sanitize skin, hair, nails, hands, etc.

Reference throughout this specification to “one embodiment”, “an embodiment”, or “a specific embodiment” means that a particular feature, structure or characteristic described in connection with the embodiment is included in at least one embodiment of the present invention and not necessarily in all embodiments. Thus, respective appearances of the phrases “in one embodiment”, “in an embodiment” or “in a specific embodiment” in various places throughout this specification are not necessarily referring to the same embodiment. Furthermore, the particular features, structures or characteristics of any specific embodiment of the present invention may be combined in any suitable manner with one or more other embodiments. It is to be understood that other variations and modifications of the embodiments of the present invention described and illustrated herein are possible in light of the teachings herein and are to be considered as part of the spirit and scope of the present invention.

While the invention is described herein in connection with certain preferred embodiments, there is no intent to limit the present invention to those embodiments. On the contrary, it is recognized that various changes and modifications to the described embodiments will be apparent to those skilled in the art upon reading the foregoing description, and that such changes and modifications may be made without departing from the spirit and scope of the present invention. Skilled artisans may employ such variations as appropriate, and the invention may be practiced otherwise than as specifically described herein. Accordingly, the intent is to cover all alternatives, modifications, and equivalents included within the spirit and scope of the invention. Moreover, any combination of the above-described elements in all possible variations thereof is encompassed by the invention unless otherwise indicated herein or otherwise clearly contradicted by context.

We claim:

1. A method for treating a surface using a cleaning substrate, the method comprising the steps of:
a) forming a cleaning substrate comprising:
i) a nonwoven web;
ii) hydrophilic materials incorporated into the nonwoven web;
ii)chemical actives incorporated into the nonwoven web;
wherein the hydrophilic materials and the chemical actives are in spatial proximity;
b) exposing a surface of the cleaning substrate to a solvent;
c) allowing the cleaning substrate to absorb the solvent; and
creating a spatial interaction between the chemical actives and the hydrophilic materials such that the hydrophilic materials compete with the chemical actives for the solvent thereby providing a controlled release of the chemical actives to the surface being cleaned.

2. The method of claim 1, wherein the solvent is water.

3. The method of claim 1, wherein the spatial interaction between the chemical actives and the hydrophilic materials occurs by having the chemical actives and the hydrophilic materials in parallel planes.

4. The method of claim 1, wherein the spatial interaction between the chemical actives and the hydrophilic materials occurs by having the chemical actives and the hydrophilic materials in a random blended proximity.

5. The method of claim 1, wherein the hydrophilic materials are selected from the group consisting of: superabsorbing polymers, silica, collagen, pectin, gelatin, starches, guar gum, gum arabic, locust bean gum, gum karaya, alginic acid, and sodium and calcium salts, and combinations thereof.

6. The method of claim 1, wherein the hydrophilic material is a synthetic substance selected from the group consisting of sodium carboxymethyl-cellulose, polyvinyl alcohol, polyvinyl pyrollidone, polyethylene glycols, crosslinked dextran, starch acrylonitrile graft copolymer, starch sodium polyacrylate, gluten, polymer of methyl vinyl ether and maleic acid and derivatives, polyvinyl pyrrolidone, polyethylene glycols, polypropylene glycols, metals and ammonium salts of polyacrylic acid, or copolymers thereof or combinations thereof.

7. The method of claim 1, further comprising a reaction between one or more chemical actives and the solvent, wherein the reaction between the one or more of the chemical actives and the solvent generates heat that can be maintained for a controlled period of time.

8. The method of claim 1, wherein the particle size of the one or more chemical actives and the hydrophilic materials may be varied to achieve the desired release profile of the chemical actives to a surface being treated.

9. A substrate providing a controlled delivery of chemical actives to a surface comprising:
a nonwoven web;
a hydrophilic material incorporated into the nonwoven web;
one or more chemical actives incorporated into the nonwoven web;
wherein a spatial interaction of the hydrophobic material and chemical actives allows the chemical actives to have a gradual release profile.

10. The substrate of claim 9, further comprising two or more layers of the nonwoven web, wherein at least one layer comprises the chemical actives and an adjacent layer comprises the hydrophilic material.

11. The substrate of claim 9, wherein the nonwoven web comprises a substantially random mixture of the chemical actives and the hydrophilic materials.

12. The substrate of claim 9, further comprising an abrasive layer.

13. The substrate of claim 9, further comprising a hi-loft layer.

14. The substrate of claim 9, wherein the spatial interaction between the chemical actives and the hydrophilic materials occurs by having the chemical actives and the hydrophilic materials in parallel planes.

15. The substrate of claim 9, wherein the hydrophilic material is a synthetic substance selected from the group consisting of sodium carboxymethyl-cellulose, polyvinyl alcohol, polyvinyl pyrollidone, polyethylene glycols, crosslinked dextran, starch acrylonitrile graft copolymer, starch sodium polyacrylate, gluten, polymer of methyl vinyl ether and maleic acid and derivatives, polyvinyl pyrrolidone, polyethylene glycols, polypropylene glycols, metals and ammonium salts of polyacrylic acid, or copolymers thereof or combinations thereof.

16. The substrate of claim 9, wherein the hydrophilic materials are selected from the group consisting of: superabsorbing polymers, silica, collagen, pectin, gelatin, starches, guar gum, gum arabic, locust bean gum, gum karaya, alginic acid, and sodium and calcium salts, and combinations thereof.

17. A substrate providing the controlled delivery of chemical actives to a surface comprising:
a nonwoven web;
a hydrophilic material incorporated into the nonwoven web;
one or more chemical actives incorporated into the nonwoven web;
wherein the spatial interaction of the hydrophobic material and chemical actives enables the substrate to give controlled, long-lasting foam delivery to the surface being treated.

18. The substrate of claim 17, further comprising two or more layers of the nonwoven web wherein at least one layer comprises the chemical actives and an adjacent layer comprises the hydrophilic material.

19. The substrate of claim 17, wherein the nonwoven web comprises a substantially random mixture of the chemical actives and the hydrophilic materials.

20. The substrate of claim 19, further comprising one or more layers of the nonwoven web which are bonded together using one of the following: thermal bonding, Through-Air-Bonding (TAB), needling, chemical bonding, point bonding, ultrasonic bonding, and combinations thereof.
